(19) United States
(12) Patent Application Publication
(10) Pub. No.: US 2003/0045841 A1
(43) Pub. Date: Mar. 6, 2003

(54) Feeding device with secondary stop tab

(76) Inventors: William M. Palcisko, Strongsville, OH (US); George J. Picha, Independence, OH (US); Davor G. Mandic, Cleveland Heights, OH (US)

Correspondence Address: PEARNE & GORDON LLP, 526 SUPERIOR AVENUE EAST, SUITE 1200, CLEVELAND, OH, 44114-1484, US

(21) Appl. No.: 09/943,864

(22) Filed: Aug. 31, 2001

(51) Int. Cl. A61M 5/00

(52) U.S. Cl. 604/256

(57) ABSTRACT

A gastrostomy feeding device having a bolster portion and a closure member. The bolster portion includes a feeding opening, a cap seat, and an anti-reflux valve and the closure member includes a secondary stop tab, which when received within the cap seat forms a seal against the anti-reflux valve.

### FIELD OF INVENTION

The present invention relates to gastrostomy feeding appliances or devices and, more particularly, to methods for sealing gastrostomy devices.

### BACKGROUND OF THE INVENTION

The present invention is an improvement of the gastrostomy feeding device of U.S. Pat. No. 6,019,746 to Picha et al, incorporated herein by reference.

Gastrostomy feeding devices generally have an internal bolster, an external bolster, a feeding conduit that extends from the external bolster to the internal bolster, and a valve that prevents the reflux of gastric fluid through the feeding conduit. Typically, a closure member is also provided to close of the feeding device during periods of non-use. These closure members generally include a cylindrical tab or stopper that is inserted into the outer open end of the feeding device. The interference fit of the stopper with the feeding device forms a seal which keeps the closure member in place.

Although a valve is provided to prevent reflux of gastric fluid through the feeding conduit, occasionally this valve is damaged while in use causing the device to leak. The closure member is then relied upon to become a secondary stop or sealing mechanism to seal off the gastric fluid until there is the opportunity to replace the feeding device. Prior art closure members are often not designed to be secondary stop tabs and may leak with less than 3 psi. of pressure.

### BRIEF SUMMARY OF THE INVENTION

This invention provides an improved closure member which can be used as a secondary stop mechanism in the event that the primary valve is damaged or becomes faulty. According to the present invention, a gastrostomy feeding device includes a bolster portion with a feeding opening, a cap seat, an anti-reflux valve, and a closure member with a secondary stop tab. The secondary stop tab fits into the cap seat and an edge or surface of the tab forms a seal against the top of the valve.

### BRIEF DESCRIPTION OF THE DRAWINGS

FIG. 1 is a front cross-sectional view of a feeding device incorporating an embodiment of the present invention;

FIG. 2 is a front elevational view of the secondary stop tab used with the feeding device shown in FIG. 1;

FIG. 3 is top plan view of the secondary stop tab shown in FIG. 2;

FIG. 4a is a front elevational view of a second embodiment of the present invention;

FIG. 4b is a front elevational view of a third embodiment of the present invention; and

FIG. 4c is a front elevational view of a fourth embodiment of the present invention;

### DETAILED DESCRIPTION OF THE INVENTION

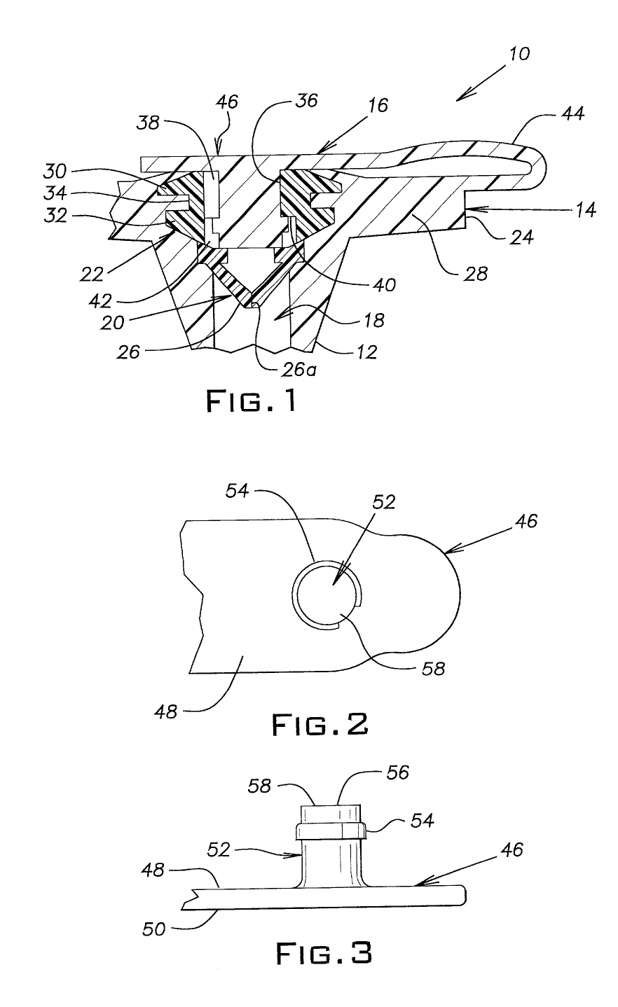

Referring now to the drawings, FIG. 1 shows a feeding device 10 incorporating an embodiment of the secondary stop tab. The feeding device consists of a feeding conduit 12, bolster portion 14, and closure member 16.

The feeding conduit 12 contains a feeding passage 18, preferably extending longitudinally the length of the feeding conduit and extending through the bolster portion. At the juncture or union of the bolster portion and the feeding conduit, the feeding passage is closed by an anti-reflux valve 20. Relatively above the anti-reflux valve 20, the feeding passage 18 is defined by a cap seat 22.

The bolster portion 14 includes the anti-reflux valve 20, a cap retainer portion 24, and the cap seat 22. The anti-reflux valve 20 may be integrally formed with the bolster portion 14 or may be separately formed and then installed into the bolster portion, as illustrated. The anti-reflux valve 20 is a duck-bill type valve and has a plurality of flexible arm members 26 that define a slit 26a and extend at an angle from the bolster portion 14 relatively beneath the cap seat 22 and into the feeding passage.

The cap retainer portion 24 includes a body portion 28 and an opening to the feeding passage 18 and provides a receptacle for the cap seat 22. The cap seat 22 is preferably a one-piece molded part that is separately formed and installed into the bolster portion 14 and includes upper and lower annular flanges 30, 32 which are joined by a cylindrical interconnecting wall 34. The cap seat 22 has a cylindrical main passageway 36 and a generally rectangular key slot 38, which provides a lock for a feeding tube (not shown). A circular recess 42 is formed in the lower wall member 32 and extends downwardly from the passageway 36. A radially inward extending upper wall of the circular recess 42 defines a bearing or support surface 40.

The closure member 16 is integrally formed with the body portion 28 and includes a flexible member 44, which has the cap retainer portion 24 on one end, and a secondary stop tab 46 on the other end. The closure member may also be separately formed from the body portion 28. As illustrated in FIGS. 2 and 3, the secondary stop tab 46 includes generally planar upper and lower base surfaces 48, 50 and a cylindrical stopper 52. The stopper 52 includes an annular flange 54 protruding radially partly or entirely around the stopper 52 and located relatively midway down the stopper 52. The stopper 52 has a flat end 58 which comprises a sealing surface 56.

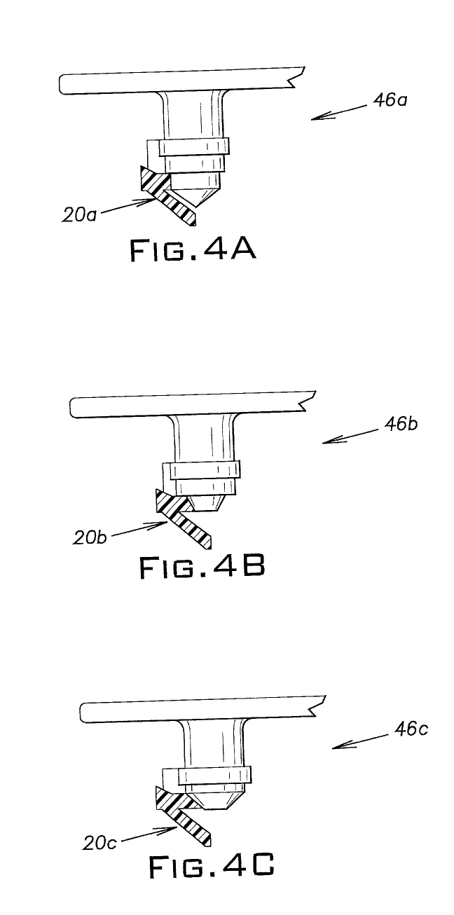

As shown in FIG. 1, the secondary stop tab 46 operates as a sealing mechanism by fitting the stopper 52 through the cap seat passageway 36 and has an interference fit with respect to the cap seat 22. The edge or surface of the flat end 58 of stopper sealing surface 56 also forms a seal against the top of the valve 20 by using the flange 54 to generate the necessary force to press the stopper against the top of the valve. Any back pressure through a defective valve 20 is resisted by the interference fit and by interengagement of the flange 54 and the support surface 40. Thus, unlike prior art closure members, which use the interference fit of the stopper and cap seat to form a seal, the secondary stop tab 46 seals by having an interference fit of the stopper and cap seat and by using the sealing surface of the stopper to press against the rim of the valve. The combination of the sealing surface and the stopper interference creates a minimum sealing pressure of 10 psi. and may withstand sealing pressures as high as 30 psi. Separate secondary stop tabs 46a-46c are shown in FIGS. 4a-4c and form alternative embodiments of the present invention. The main modification to the previously described closure member is the fact that the sealing surface 56 of the secondary stop tab can have a variety of profiles arising from the flat end 58 depending on the shape of the surface it is sealing against.

While the invention has been described with reference to a specific embodiment, various changes may be made and equivalents may be substituted for elements thereof by those skilled in the art without departing from the scope of the invention. In addition, other modifications may be made to adapt a particular situation or material to the teachings of the invention without departing from the essential scope thereof. The present invention herein is not to be construed as being limited, except insofar as indicated in the appended claims.

What is claimed is:

1. A gastrostomy feeding device comprising: a bolster portion and a closure member; the bolster portion includes a feeding opening, a cap seat, and an anti-reflux valve; the cap seat being disposed relatively between the feeding opening and the anti-reflux valve; the closure member including a secondary stop comprising a stopper received within the cap seat; and the stopper forms an interference fit with the cap seat and the stopper forms a seal against the anti-reflux valve.

2. An apparatus as in claim 1 wherein the stopper is provided with at least one flange protruding radially at least partly around the stopper, and the flange axially bears against a portion of the cap seat and presses the stopper against a top portion of the anti-reflux valve.

3. An apparatus as in claim 2 wherein the secondary stop includes a flat sealing surface at the end of the stopper, and an edge of the flat surface seals against the top portion of the anti-reflux valve.

4. An apparatus as in claim 3 wherein a frustoconical portion is formed on the flat sealing surface.

5. An apparatus according to claim 3 wherein a cylindrical portion is formed on the flat sealing surface.

6. An apparatus as in claim 3 wherein the closure member is integrally formed with the bolster portion and includes a flexible member.

7. A gastrostomy feeding device comprising:
a bolster, a feeding conduit extending from the bolster portion, and a closure member; the bolster portion includes an anti-reflux valve, and a cap seat, and a feeding opening; the cap seat being disposed relatively between the anti-reflux valve and feeding opening; the closing member includes a secondary stop comprising a stopper which is received within the cap seat; and the stopper is provided with at least one flange protruding radially around the stopper, wherein the flange axially bears against a portion of the cap seal and presses the stopper against a top portion of the anti-reflux valve, and a edge or surface of the stopper forms a seal against the anti-reflux valve.
